(19) United States
(12) Patent Application Publication
(10) Pub. No.: US 2011/0090452 A1
(43) Pub. Date: Apr. 21, 2011

(54) EYEGLASS USING ADJUSTABLE PUPIL MASK TO IMPROVE VISION

(76) Inventor: Charles J. Kulas, San Francisco, CA (US)

(21) Appl. No.: 12/580,236

(22) Filed: Oct. 15, 2009

(51) Int. Cl. G02C 7/16; A61B 3/00

(52) U.S. Cl. 351/45; 351/246

(57) ABSTRACT

An apparatus for adjusting the amount of light passing through a pupil of a human eyeball particularly for a person having defects or imperfections in the eyeball lens. An electrically controllable mask area having a smallest, a medium and a largest mask opening may be coupled to at least one lens of the eyeglass. The mask area is varied with a rotating aperture adjustment assembly on a controller so that an image of an object that would otherwise pass through the pupil is masked so that a smaller portion of the image passes through the pupil. The wearer may select the at least one mask opening by moving a thumb wheel on the rotating aperture adjustment assembly. The wearer selects at least one of on and off condition of the apparatus with a button. The mask area can be customized by means of an external processing system.

### CROSS REFERENCES TO RELATED APPLICATIONS

This application is related to co-pending U.S. patent application Ser. No. [TBD] filed on Oct. 15, 2009, entitled “EYEGLASS INCLUDING A LIGHT SOURCE DIRECTED AT THE EYE” which is hereby incorporated by reference as if set forth in full in this specification for all purposes.

### BACKGROUND

Embodiments of the invention relate generally to an eyeglass for improving the vision and more specifically to an eyeglass using an adjustable pupil mask.

Due to effects such as genetics, the environment, old age, etc., a person's eye lens may become imperfect as it loses its desired shape or curvature, becomes damaged, tainted or internally obscured, or suffers from other undesirable effects, defects or errors. When an iris is contracted, the pupil, which is the opening of the eye, becomes tiny and a smaller part of the lens is used. Hence, the error may be diminished and can become inconsequential or negligible if the part of the lens in error is no longer being used to process an image that enters the smaller pupil.

Such contracting of the iris occurs naturally, in response to the presence of a greater amount of light. However, controlling the iris size by controlling ambient lighting is often not possible or practical and in many cases may only achieve a limited improvement.

### SUMMARY

Embodiments of the present invention provide an apparatus for adjusting the amount of an image passing through a pupil of a human eyeball. The apparatus may be useful to a person having defects or imperfections in the eyeball lens. The apparatus can compensate for the defects by restricting the light rays from an image to pass through a smaller portion of the pupil to reach the retina. The light rays entering the pupil can be concentrated around the center thereof and hence errors in vision can be reduced.

An electrically controllable mask area is placed on a front surface, a back surface or embedded within the at least one lens of the eyeglass. The mask area includes a plurality of mask openings such as a largest mask opening, a medium mask opening and a smallest mask opening. The mask area may be varied in response to an activation of a control by at least one user and an external signal received by the controller. A button on the controller permits the wearer to select at least one condition of the apparatus, the at least one condition may be an on and off. The wearer selects the at least one mask opening by moving a thumb wheel on a rotating aperture adjustment assembly.

An alternate embodiment of the invention includes a sensor coupled to an eyeglass worn by a wearer. The light sensor senses the ambient light condition and sends signals to a controller that facilitates to adjust the size of the mask openings in response to the received signal. The largest mask opening corresponds to the maximum dilated pupil size thereby permitting light rays to impinge upon the maximum area of the pupil and the smallest mask opening restricts light rays to impinge upon a less portion of the pupil to reach retina.

The mask area can be customized by means of an external processing system with a wired and/or a wireless communication means. The wearer is positioned with regard to a display associated with the external processing system and a recommended number of mask openings and shape of the mask opening may be loaded in the controller of the apparatus. At least one specification may be obtained for a clear view of at least one pattern on a display of the external processing system by the wearer and the specification that is obtained can be loaded in the controller.

One embodiment provides an apparatus for adjusting the amount of light passing through a pupil of a human eyeball, the apparatus comprising an eyeglass having a lens; at least one electrically controllable mask area fixedly coupled to the lens of the eyeglass; and at least one controller for varying the mask area so that an image of an object that would otherwise pass through the pupil is masked so that a smaller portion of the image passes through the pupil.

A further understanding of the nature and the advantages of particular embodiments disclosed herein may be realized by reference of the remaining portions of the specification and the attached drawings.

### BRIEF DESCRIPTION OF THE DRAWINGS

FIG. 1A is a schematic view of an apparatus for adjusting the amount of light passing through a pupil of a human eyeball having an electrically controllable mask area adapted to arrange on at least one lens of an eyeglass and illustrating the optical relationship between a largest mask opening and the pupil of the human eyeball during low light condition;

FIG. 1B is a blow-up schematic view of FIG. 1A wherein the optical relationship between the largest mask opening and the pupil of the human eyeball is illustrated during low light condition;

FIG. 1C is a schematic view of the electrically controllable mask area adapted to at least one lens of the eyeglass worn by the wearer and illustrating the optical relationship between a smallest mask opening and the pupil of the human eyeball during high light condition;

FIG. 1D is a blow-up schematic view of FIG. 1C wherein the optical relationship between the smallest mask opening and the pupil of the human eyeball is illustrated during high light condition;

FIG. 2 illustrates a front view of the electrically controllable mask area having a plurality of concentric circular mask openings and a controller coupled to the electrically controllable mask area;

FIG. 3 illustrates a front view of the electrically controllable mask area having at least one non-circular mask openings and the controller coupled to the electrically controllable mask area;

FIG. 4 is a schematic view of another embodiment of the present invention wherein a light sensor is coupled to an eyeglass worn by a wearer and illustrating the optical relationship between a medium mask opening and a pupil of a human eyeball during medium light condition;

FIG. 5 illustrates a schematic view of a processing system having a wireless communication with the controller of the preferred embodiment of the present invention;

FIG. 6 shows a schematic view the eyeglass of the preferred embodiment of the present invention wherein at least one eye of the wearer looking on a plurality of radial arms of characters on at least one display of the processing system;

FIG. 7 shows a blow-up view of the plurality of radial arms of characters of FIG. 6 on at least one display of the processing system;

FIG. 8 shows a block diagram of a controller of the preferred embodiment of the present invention for processing the signals from at least one user control; and

FIG. 9 shows an operational flowchart illustrating a method for calibrating a mask pattern by adjusting the amount of light passing through the pupil of the human eyeball.

### DETAILED DESCRIPTION OF EMBODIMENTS

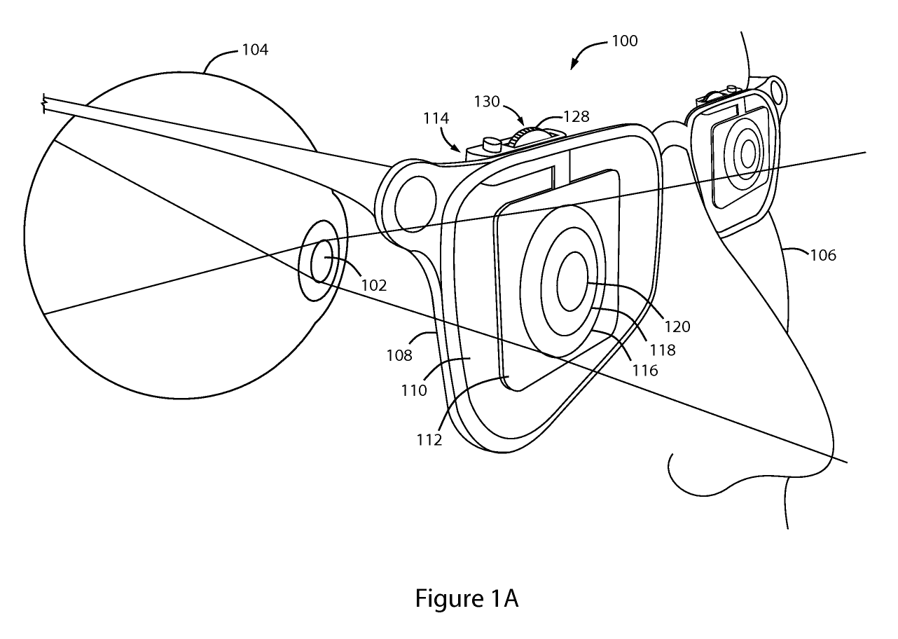

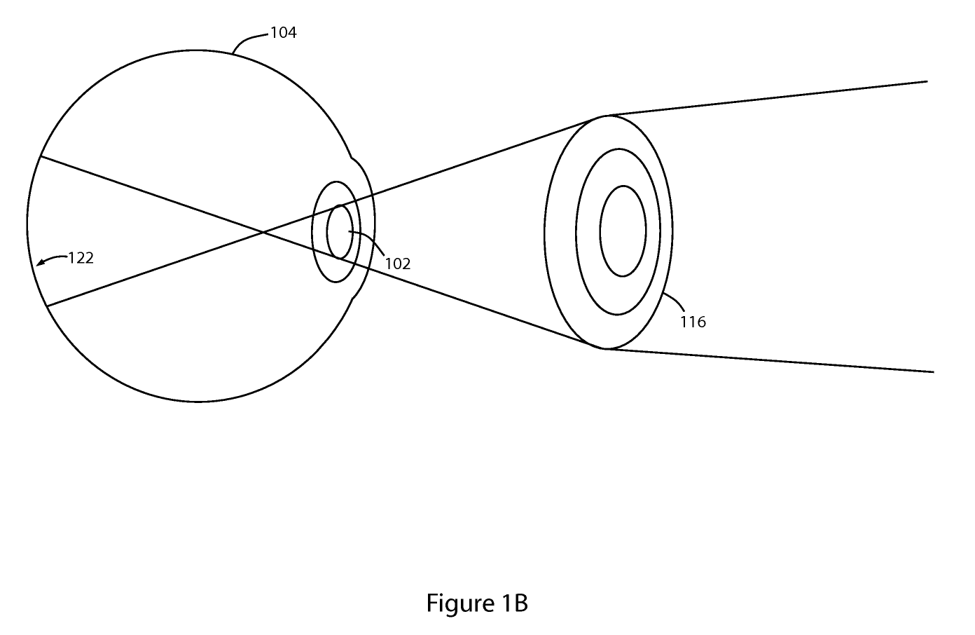

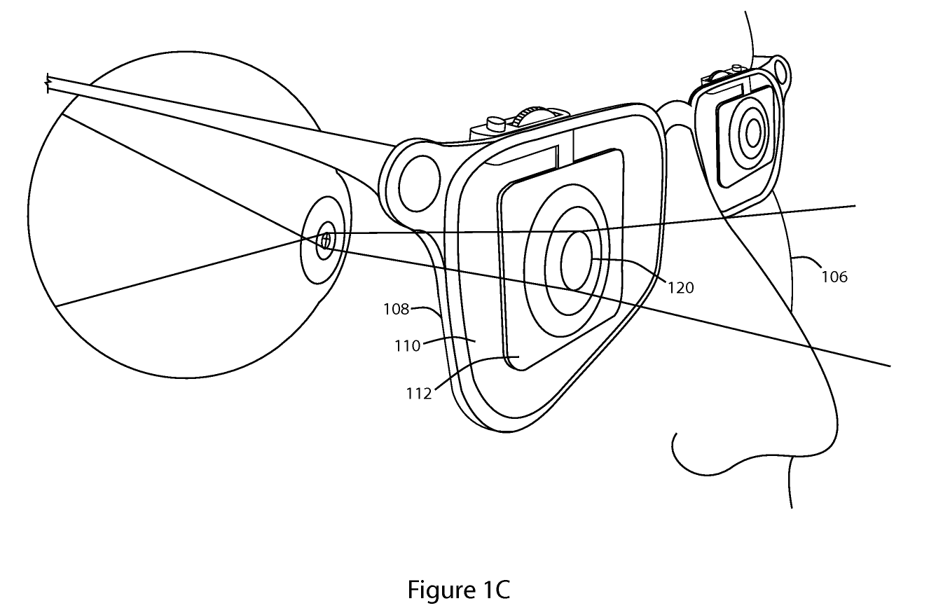

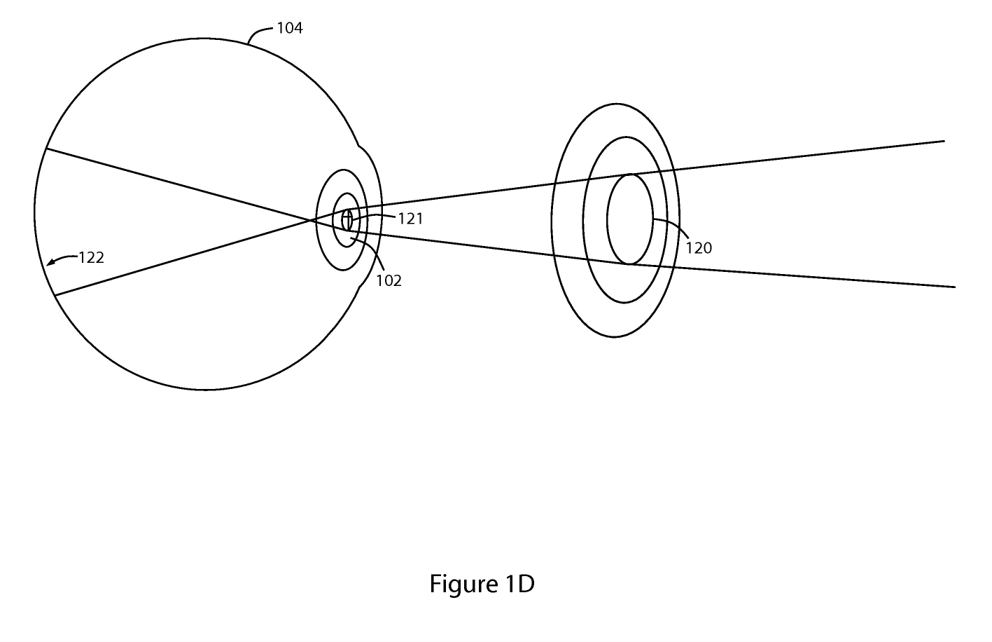

FIG. 1A is a schematic view of an apparatus 100 for adjusting the amount of light passing through a pupil 102 of a human eyeball 104 having an electrically controllable mask area 112 adapted to arrange on at least one lens 110 of an eyeglass 108 illustrates the optical relationship between a largest mask opening 116 and the pupil 102 of the human eyeball 104 during low light condition. The apparatus 100 may be shown worn by a wearer 106. The apparatus 100 includes an eyeglass 108 having a lens 110. At least one electrically controllable mask area 112 is fixedly coupled to at least one lens 110 of the eyeglass 108. The preferred embodiment further includes at least one controller 114 for varying the mask area 112 so that an image (not shown) of an object (not shown) that would otherwise pass through the pupil 102 is masked so that a smaller portion of the image passes through the pupil 102. Variation of the mask area 112 can be in response to at least one of the wearer's 106 activation of a control and/or an external signal received by the controller 114. A thumb wheel 128 on a rotating aperture adjustment assembly 130 in the controller 114 may be utilized to select a mask opening such as a medium mask opening 118 or a smallest mask opening 120 in addition to the largest mask opening 116. Although specific sizes of masks are illustrated, a preferred embodiment allows the mask size to be continuously varied. In other words, many different mask sizes can be selected including intermediary sizes to those shown in FIG. 1A.

FIG. 1B is a blow-up schematic view of FIG. 1A wherein the optical relationship between the largest mask opening 116 and the pupil 102 of the human eyeball 104 is illustrated during low light condition. The largest mask opening 116 corresponds to the maximum dilated pupil size thereby permitting light rays to impinge upon the maximum area of the pupil 102 to reach retina 122. Since the largest mask opening 116 permits all of the fully dilated pupil to receive image information, the largest mask does not, in fact, mask any of the image from the wearer's vision. Larger mask openings can be used but they will not result in any more of the image impinging on the wearer's retina.

FIG. 1C is a schematic view of the electrically controllable mask area 112 adapted to arrange on at least one lens 110 of the eyeglass 108 worn by the wearer 106 for illustrating the optical relationship between the smallest mask opening 120 and the pupil 102 of the human eyeball 104 during high light condition.

FIG. 1D is a blow-up schematic view of FIG. 1C wherein an optical relationship between the smallest mask opening 120 and the pupil 102 of the human eyeball 104 is illustrated during high light condition. The smallest mask opening 120 restricts light rays to impinge upon a less portion of the pupil 102 to reach the retina 122. The light rays entering the pupil 102 concentrate around the center 121 thereof and the probability of error caused on the image of the object that impinges upon the defective eye lens (not shown) becomes less.

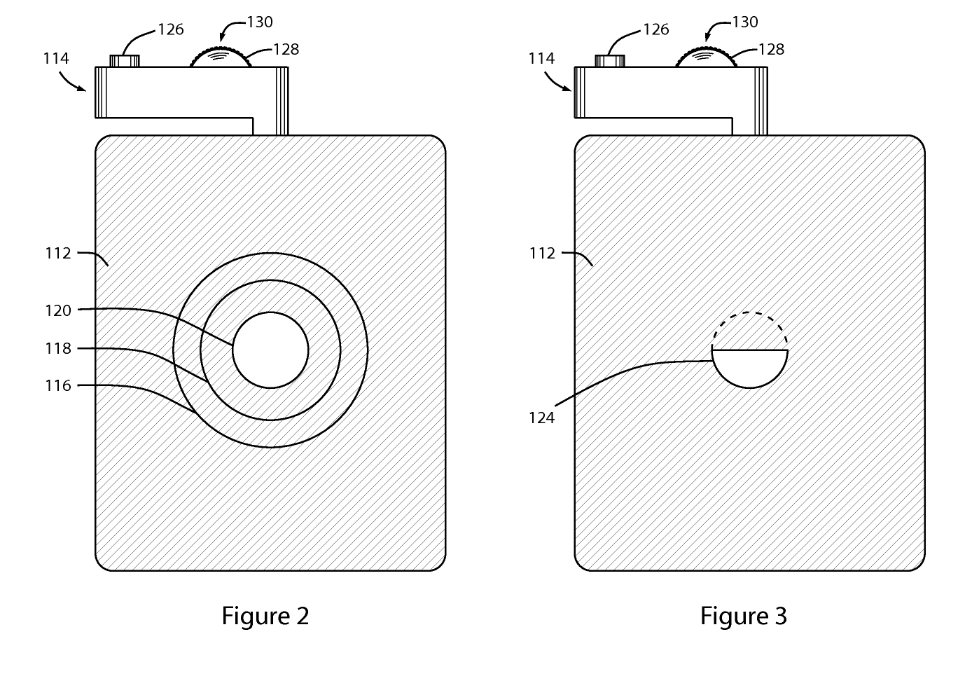

FIGS. 2 and 3 illustrate the schematic view of the controller 114 that is coupled to the electrically controllable mask area 112. The mask area of FIG. 2 includes concentric circular mask openings such as the largest mask opening 116, the medium mask opening 118 and the smallest mask opening 120. With reference to FIG. 3, the mask area 112 includes a non-circular mask opening 124. In general, the mask area 112 can have any number of mask openings. The mask area 112 may be placed on a front surface, a back surface or embedded within the at least one lens 110 of the eyeglass 108. The mask area 112 is opaque and the at least one selected mask opening 116, 118, 120 is transparent. A button 126 on the controller 114 permits the wearer 106 to select at least one of on and off condition of the apparatus 100. The wearer 106 can select the at least one mask opening 116, 118, 120 by moving the thumb wheel 128 on the rotating aperture adjustment assembly 130. The mask area 112 may be made of any suitable kind of material such as a liquid crystal display or the like.

Although preferred embodiments of the invention contemplate using the mask in association with eyeglasses having lenses that are designed to correct vision, other embodiments can be used apart from vision correcting lenses. Thus, for purposes of this specification, the term “lens area” includes optically flat transmissive material (e.g., glass, plastic, etc.) that may not be designed to correct for vision defects. In other cases a lens area can include a through hole or opening that is fully or partially framed by a structure that is supported in proximity to the eye. For example, a mask may be supported by a structure that resembles an eyeglass with all or a part of the traditional eyeglass lens removed. In FIG. 1, for example, lens 110 may be omitted so that the lens area occupied by lens 110 is left empty to create a space. Mask area 110 can be formed on any desired transmissive material and can be supported by any suitable coupling to the eyeglass frame such as the coupling to controller 114 which is, in turn, affixed to eyeglass 108. Other present and future designs for supporting a mask area adjacent to an eye are possible and may be within the scope of the claims.

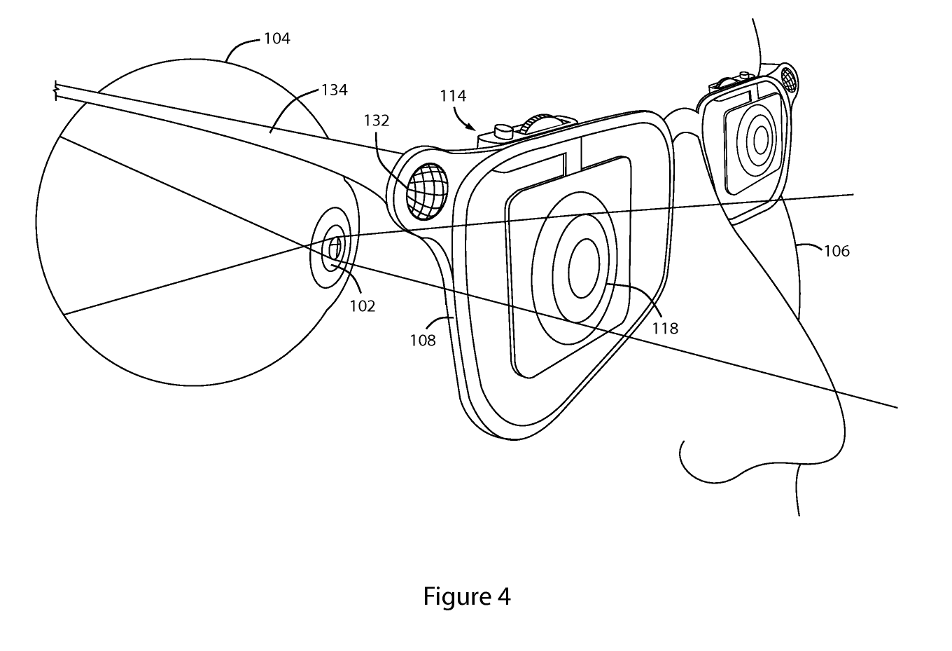

FIG. 4 is another embodiment of the present invention illustrating a light sensor 132 coupled to an eyeglass 108 worn by a wearer 106. FIG. 4 also shows the optical relationship between a medium mask opening 118 and a pupil 102 of a human eyeball 104 during medium light condition. The light sensor 132 senses the outside light condition and sends signals to a controller 114 that facilitates to adjust the size of the mask openings 116, 118, 120 in response to the received signal. The sensor 132 that may be located in the proximal end of an eyeglass arm 134 may be a proximity sensor. Hence, when an object such as a book is held up close to the eye, automatic masking may be triggered which can be a combination of the response of the outside light condition and the proximity of the object right in front of the eyes. The eyeglass 108 can take various designs, for example, custom type holders resembling the eyeglass 108 can be used for supporting the LCD display. Such a design need not have the lens.

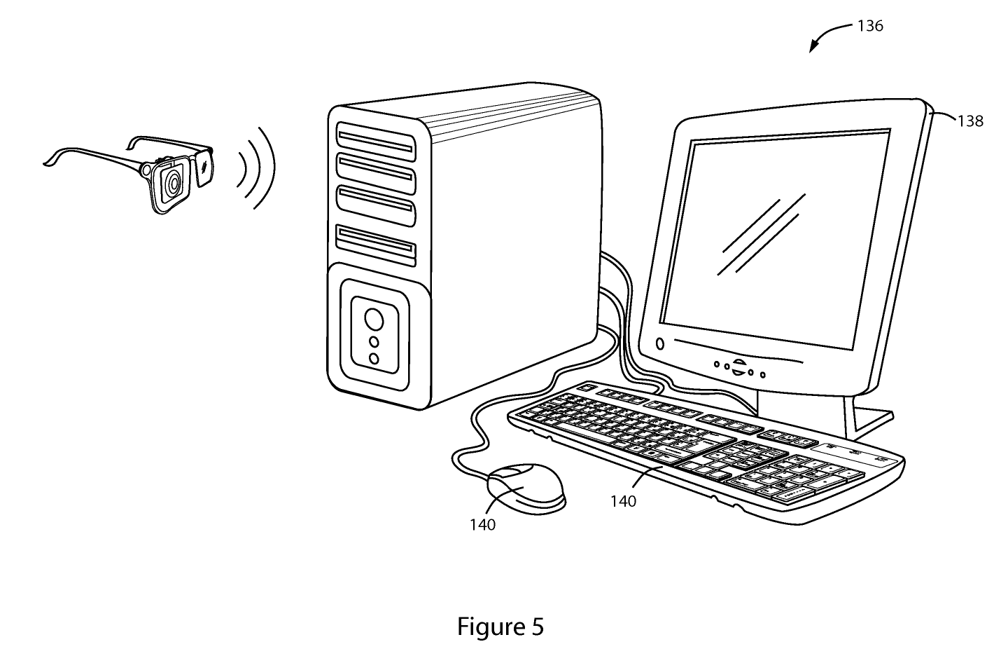

FIG. 5 is a schematic view of a processing system 136 having a wireless communication with the controller 114 of the preferred embodiment of the present invention. The mask area 112 of the apparatus 100 can be customized by means of the external processing system 136. The external processing system 136 can be a laptop, a notepad, a sub not book, desktop, cell phone, game devise, etc. In general, the external processing system 136 can be any suitable processing system with processing abilities that has a display 138, a user input device 140 and probably adaptable for use with the embodiments of this invention. Any suitable communication approach may be employed between the controller 114 and the processing system 136 such as wired, wireless (radio frequency, infrared, etc.), optical, acoustic, or the like.

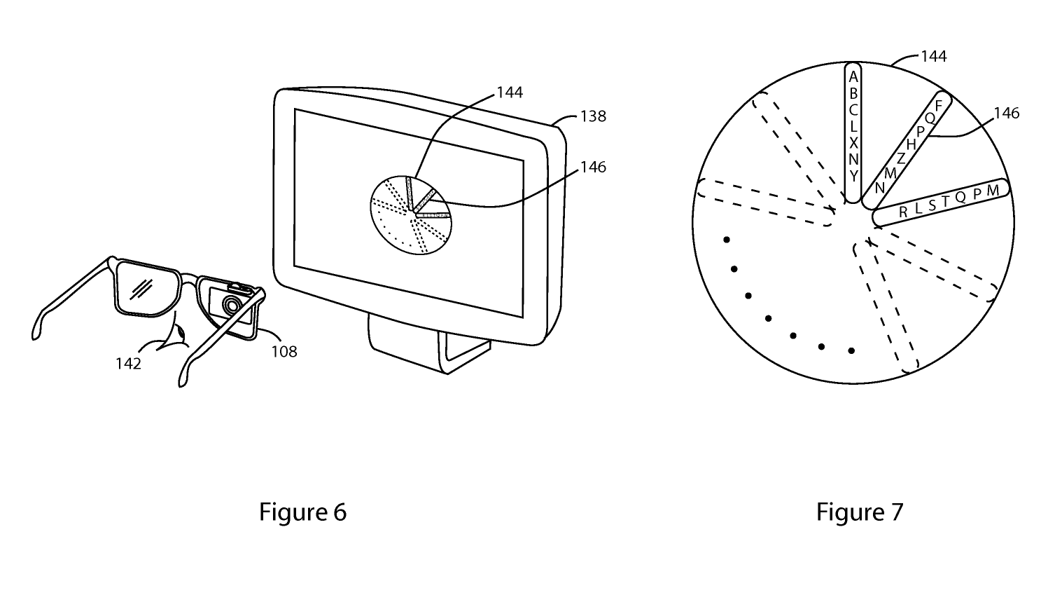

FIG. 6 is a schematic view of the eyeglass of the preferred embodiment of the present invention wherein at least one eye 142 of the wearer 106 looking through the eyeglass 108 on a plurality of radial arms of characters 144 on the display 138 of the processing system 136. The external processing system 136 can load the recommended number of mask openings 116, 118, 120 and the shape of the mask opening 116, 118, 120 in the controller 114 of the apparatus 100. The wearer's visual clarity can be determined by selecting at least one pattern 146 on the display 138 with a control (not shown) in the external processing system 136. The wearer 106 can position themselves closer or farther from the display 138 and the display 138 can provide different intensities of light, different sizes of the image pattern where any sort of pattern can be used, such as an array of lines, an art chart or a picture such as a geometric design, a face, etc. to determine visual clarity. Once the user's specific vision characteristics are determined, one or more corresponding mask designs can be stored in the controller 114 or stored in other devices a such as a desktop, a cell phone or the like and loaded into the controller 114 with the communication medium, for example, Bluetooth communication, USB wired communication or any other suitable wired or wireless transfer.

FIG. 7 shows a blow-up view of the plurality of radial arms of characters 144 of FIG. 6 on at least one display 138 of the processing system 136 of the preferred embodiment of the present invention. There may be a plurality of different radial arms of characters, for example 36. The wearer 106 can select at least one radial arms of characters 146 for determining the visual clarity.

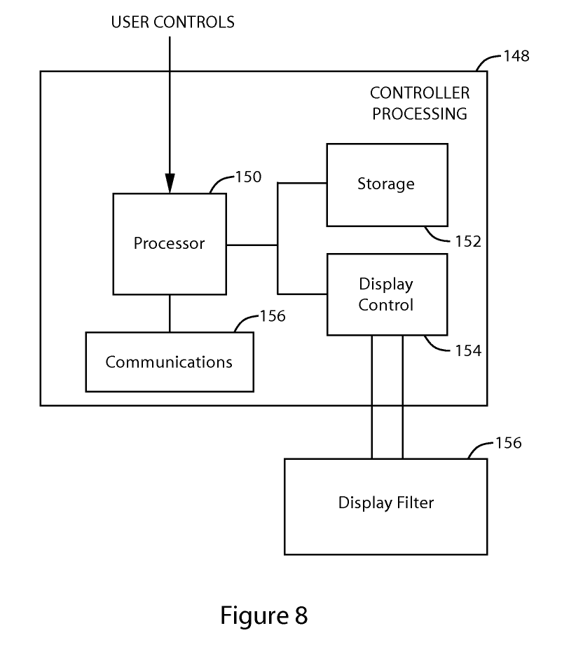

FIG. 8 shows a block diagram of the controller for processing 148 the signals from at least one user control of the preferred embodiment of the present invention. A processor 150 of the present embodiment is associated with a plurality of subsystems including user controls such as the push button 126 and the thumb wheel 128, a storage memory 152, a display control 154 and a means for communication 156. A power source (not shown) provides sufficient power to each subsystem in the controller 114. The user controls may be a touch type, voice recognition control or the like. The means for communication 156 in connection with the processor 150 may be used for accessing wired or wireless communication with a USB cable, Bluetooth or wi-fi, wireless Internet or any such type of wireless connection. Any type of communication is possible that allows connecting with a computer or an external processor, for example a cell phone or any such device that could go over the Internet to an optician's office. One can download different mask patterns and the mask patterns can be stored in the storage memory 152 such as a non-volatile Random Access Memory or any suitable types of storage memory. The wearer 106 can also store the settings manually in the storage memory 152. The display control 154 is used to control a display filter 156 that may be an LCD display. The display control 154 can be mounted anywhere on the eyeglasses 108. The display control 154 can be somebody's cell phone, a watch, under eye pad or any thing that can communicate with the controller for processing 148.

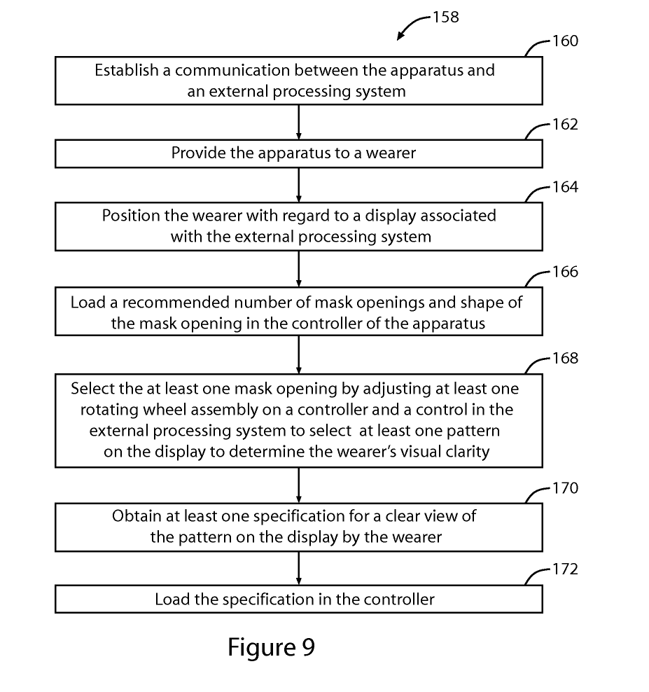

FIG. 9 shows an operational flowchart that illustrates a method for calibrating a mask pattern of an apparatus for improving vision 158 by adjusting the amount of light passing through a pupil of a human eyeball. As indicated in block 160, a communication is established between the apparatus and an external processing system. The apparatus is provided to a wearer as indicated in block 162. The wearer is positioned with regard to a display associated with the external processing system as indicated in block 164. A recommended number of mask openings and shape of the mask opening may be loaded in the controller of the apparatus as indicated in block 166. At least one mask opening is selected by adjusting at least one rotating wheel assembly on a controller and a control in the external processing system to select at least one pattern on the display to determine the wearer's visual clarity as indicated in block 168. At least one specification is obtained for a clear view of the pattern on the display by the wearer as indicated in block 170. The specification that is obtained is loaded in the controller as indicated in block 172.

Although the description has been described with respect to particular embodiments thereof, these particular embodiments are merely illustrative, and not restrictive. For example, although embodiments of the invention have been described primarily with respect to changing the mask opening with the controller, other mechanisms may be employed for varying the mask area in response to an external signal, or to improve vision of a wearer. In a simplified embodiment, the mask opening may be created by inserting, pasting or otherwise affixing or placing in proximity a separate piece of material onto or next to an eyeglass. The material may be an opaque piece of tape or paper with a small hole. They eyeglass can be provided with a holder for the material such that the material can be affixed to the eyeglass.

Any suitable programming language can be used to implement the routines of particular embodiments including C, C++, Java, assembly language, etc. Different programming techniques can be employed such as procedural or object oriented. The routines can execute on a single processing device or multiple processors. Although the steps, operations, or computations may be presented in a specific order, this order may be changed in different particular embodiments. In some particular embodiments, multiple steps shown as sequential in this specification can be performed at the same time.

Particular embodiments may be implemented in a computer-readable storage medium for use by or in connection with the instruction execution system, apparatus, system, or device. Particular embodiments can be implemented in the form of control logic in software or hardware or a combination of both. The control logic, when executed by one or more processors, may be operable to perform that which is described in particular embodiments.

Particular embodiments may be implemented by using a programmed general purpose digital computer, by using application specific integrated circuits, programmable logic devices, field programmable gate arrays, optical, chemical, biological, quantum or nanoengineered systems, components and mechanisms may be used. In general, the functions of particular embodiments can be achieved by any means as is known in the art. Distributed, networked systems, components, and/or circuits can be used. Communication, or transfer, of data may be wired, wireless, or by any other means.

It will also be appreciated that one or more of the elements depicted in the drawings/figures can also be implemented in a more separated or integrated manner, or even removed or rendered as inoperable in certain cases, as is useful in accordance with a particular application. It is also within the spirit and scope to implement a program or code that can be stored in a machine-readable medium to permit a computer to perform any of the methods described above.

As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise.

Thus, while particular embodiments have been described herein, latitudes of modification, various changes, and substitutions are intended in the foregoing disclosures, and it will be appreciated that in some instances some features of particular embodiments will be employed without a corresponding use of other features without departing from the scope and spirit as set forth. Therefore, many modifications may be made to adapt a particular situation or material to the essential scope and spirit.

1. An apparatus for adjusting the amount of light passing through a pupil of a human eyeball, the apparatus comprising:
an eyeglass having a lens;
at least one electrically controllable mask area fixedly coupled to the lens of the eyeglass;
at least one controller for varying the mask area so that an image of an object that would otherwise pass through the pupil is masked so that a smaller portion of the image passes through the pupil.

2. The apparatus of claim 1, wherein varying the mask area is in response to a user's activation of a control.

3. The apparatus of claim 1, wherein varying the mask area is in response to an external signal received by the controller.

4. The apparatus of claim 1, wherein the at least one mask opening may be a largest mask opening, a medium mask opening and a smallest mask opening.

5. The apparatus of claim 1, wherein the mask area is placed on at least one of a front surface, a back surface, or embedded within the at least one lens of the eyeglass.

6. The apparatus of claim 1, wherein the mask area is opaque and the at least one mask opening is transparent.

7. The apparatus of claim 1, wherein the largest mask opening corresponds to the maximum dilated pupil size permitting light rays to impinge upon the maximum area of the pupil to reach retina.

8. The apparatus of claim 1, wherein the smallest mask opening restricts light rays to impinge upon a less portion of the pupil to reach retina.

9. The apparatus of claim 7, wherein the light rays entering the pupil concentrates around the center thereof.

10. The apparatus of claim 1, wherein the controller allows the wearer to select at least one condition of the apparatus.

11. The apparatus of claim 10, wherein the controller allows the to select a size of the mask opening.

12. The apparatus of claim 1, wherein the mask area includes a liquid crystal display.

13. The apparatus of claim 1, wherein a mask design is customizable by means of an external processing system.

14. The apparatus of claim 13, wherein the external processing system is configured to communicate with the controller by means of a wired and/or a wireless communication means.

15. The apparatus of claim 14, wherein the external processing system includes a computing system.

16. The apparatus of claim 1, wherein the lens includes an optically flat area.

17. The apparatus of claim 1, wherein the lens includes a through hole opening.

18. A method for calibrating a mask pattern for improving vision by adjusting the amount of light passing through a pupil of a human eyeball, the method comprising:
a processing system including a display and a processor;
communicating from the processor to an eyeglass, wherein the eyeglass includes a mask opening;
displaying one or more patterns on the display;
determining a person's visual clarity of a pattern on the display; and
adjusting the mask opening in response to the determination of a person's visual clarity.

19. The method of claim 14, wherein one or more mask opening patterns are loaded into a controller on the eyeglass.
